(19) United States
(12) Patent Application Publication
(10) Pub. No.: US 2015/0329878 A1
(43) Pub. Date: Nov. 19, 2015

(54) FERMENTATIVE PRODUCTION OF A HYDROCARBON

(71) Applicant: GLOBAL BIOENERGIES, Evry (FR)

(72) Inventor: Richard Bockrath, Wilmington, DE (US)

(21) Appl. No.: 14/650,523

(22) PCT Filed: Dec. 3, 2013
(86) PCT No.: PCT/EP2013/075393

§ 371 Date: Jun. 8, 2015

(30) Foreign Application Priority Data
Dec. 7, 2012 (EP) 12196056.1

(51) Int. Cl. C12P 5/02; C12P 5/00

(52) U.S. Cl. CPC C12P 5/026; C12P 5/007

(57) ABSTRACT

The present invention relates to a process for the fermentative production of a hydrocarbon, wherein a microorganism producing the hydrocarbon is cultured in a liquid fermentation medium in a fermenter, wherein an inlet gas comprising oxygen is fed into the fermenter and the total pressure of the inlet gas before introduction into the fermenter is about 1.5 bar to about 15 bar (about 150 kPa to about 1500 kPa), wherein the hydrocarbon is obtained in a gaseous state in the fermentation off-gas, and wherein the concentration of oxygen in the fermentation off-gas is controlled to be below about 10 vol-%. The process is particularly cost effective, eliminates or reduces the risk of inflammation of the fermentation off-gas, and facilitates the isolation of the hydrocarbon from the fermentation off-gas.

The present invention relates to a process for the fermentative production of a hydrocarbon, such as isobutene, using a microorganism cultured in a fermenter.

Fermentative production processes play an important role in the provision of various hydrocarbons and often constitute an important alternative to chemical processes. Since the operating costs of fermentation processes can be immense, there is a constant need to provide more cost efficient processes. Attempts to decrease operating costs include, e.g., the development of recombinant organisms which use a metabolic pathway for the production of the desired compound showing a reduced energy consumption, development of enzymes with higher activities, increasing efficiency in substrate conversion, the use of cheaper substrates and the like. Over the last decade attempts to produce hydrocarbon compounds which are of interest for the industry (e.g., as biofuels or as components of polymers) with the help of fermentation processes have increased and in the meantime various enzymatic reactions for the production of such compounds in microorganisms have been described. The development of corresponding fermentation methods involves its own problems since they often make use of completely new metabolic pathways involving unusual enzymatic conversions. The fermentative production of certain hydrocarbons is described, e.g., in van Leeuwen B N et al., Appl Microbiol Biotechnol, 2012, 93(4):1377-1387 and in WO 2012/052427. The effects of pressure on bacteria and corresponding biotechnological applications are furthermore described in Follonier S et al., Appl Microbiol Biotechnol, 2012, 93(5):1805-1815.

The present invention addresses the need to provide an improved method for the fermentative production of a hydrocarbon. In particular, the process of the invention is advantageous in that it allows for an elimination or reduction of the risk of combustion of the fermentation off-gas which contains the desired gaseous hydrocarbon in combination with oxygen and is thus potentially inflammable. The process of the invention furthermore allows for an improved production efficiency and an improved yield of the desired hydrocarbon (in relation to the amount of oxygen fed into the fermenter), and the facilitation of the subsequent isolation of the hydrocarbon from the fermentation off-gas.

Accordingly, the present invention provides a process for the fermentative production of a hydrocarbon, wherein a microorganism producing the hydrocarbon is cultured in a liquid fermentation medium in a fermenter, wherein an inlet gas comprising oxygen is fed into the fermenter and the total pressure of the inlet gas before introduction into the fermenter is about 1.5 bar to about 15 bar (about 150 kPa to about 1500 kPa), wherein the hydrocarbon is obtained in a gaseous state in the fermentation off-gas, and wherein the concentration of oxygen in the fermentation off-gas is controlled to be below about 10 vol-%.

In the process provided herein, fermentation takes place at hyperbaric pressure. In particular, the total pressure of the inlet gas before introduction into the fermenter is about 1.5 bar to about 15 bar (about 150 kPa to about 1500 kPa). Preferably, it is about 1.5 bar to about 10 bar (about 150 kPa to about 1000 kPa). It may also be, e.g., about 2 bar to about 10 bar (about 200 kPa to about 1000 kPa). More preferably, the total pressure of the inlet gas before introduction into the fermenter is about 1.5 bar to about 8 bar (about 150 kPa to about 800 kPa), even more preferably it is about 1.5 bar to about 6 bar (about 150 kPa to about 600 kPa), even more preferably it is about 2 bar to about 6 bar (about 200 kPa to about 600 kPa), even more preferably it is about 3 bar to about 6 bar (about 300 kPa to about 600 kPa), and yet even more preferably it is about 3.5 bar to about 6 bar (about 350 kPa to about 600 kPa).

The inlet gas is typically fed directly into the liquid fermentation medium, preferably through a feed orifice at the bottom of the fermenter, and passes through the liquid medium into the headspace of the fermenter (i.e., the gas phase above the liquid fermentation medium inside of the fermenter), from where the fermentation off-gas is retrieved. While the pressure of the headspace gas in the fermenter will be approximately the same as the pressure of the inlet gas before introduction, the pressure of the liquid fermentation medium (and of the gas phase dispersed in the liquid fermentation medium) will further depend on the depth of the liquid (i.e., the height of the liquid medium in the fermenter) and the density of the liquid medium. The liquid fermentation medium and the gas phase dispersed in the liquid fermentation medium will consequently have a pressure gradient, showing a higher pressure towards the bottom of the fermenter.

If, for example, a fermenter having a height of liquid fermentation medium of 20 m is used, the total pressure at the bottom of the fermenter will be about 2 bar (about 200 kPa) plus the pressure of the headspace gas above the liquid fermentation medium. In that case, if an inlet gas having a pressure before introduction into the fermenter of about 4 bar (about 400 kPa) is fed into the liquid fermentation medium at the bottom of the fermenter, the pressure of the inlet gas streaming in at a liquid depth of 20 m will be approximately 6 bar (approximately 600 kPa) while it will be approximately 5 bar (approximately 500 kPa) at a liquid depth of 10 m and about 4 bar (about 400 kPa) in the headspace of the fermenter.

During the course of fermentation, the pressure in the liquid fermentation medium may slightly change as a result of the growth of the microorganism, its consumption of nutrients and its excretion of metabolic products. The inlet gas will flow through the liquid fermentation medium into the headspace of the fermenter, from where the fermentation off-gas will be obtained. The pressure of the headspace gas and, accordingly, the pressure of the fermentation off-gas will correspond essentially to the pressure of the inlet gas before introduction into the fermenter but may slightly vary therefrom, e.g., due to the consumption of oxygen comprised in the inlet gas by the microorganism as well as the emission of gaseous metabolic products of the microorganism, including the desired hydrocarbon and possibly further gaseous substances produced by the microorganism (such as carbon dioxide).

The increased pressure of the inlet gas before introduction into the fermenter of about 1.5 bar to about 15 bar (about 150 kPa to about 1500 kPa) is advantageous as it results in an increased transfer of oxygen comprised in the inlet gas into the liquid fermentation medium, which in turn allows for an improved oxygen utilization by the microorganism. The improved oxygen utilization considerably decreases the concentration of oxygen in the fermentation off-gas and thereby facilitates controlling the oxygen concentration in the fermentation off-gas to be below about 10 vol-%. This is advantageous as it eliminates or greatly reduces the risk of combustion of the fermentation off-gas, as explained in more detail further below. The increased pressure of the inlet gas thus contributes to the safety improvement with respect to flammability achieved by the process of the present invention.

Due to the improved oxygen utilization by the microorganism resulting from the increased pressure of the inlet gas, an improved production efficiency and an improved yield of the desired hydrocarbon (relative to the amount of oxygen fed into the fermenter) can further be achieved, particularly since the oxygen supply is often a limiting factor in fermentative production processes. Moreover, increased utilization of the oxygen fed into the fermenter will also decrease the total volume of the oxygen-carrying inlet gas required and will result in smaller volumes of gas to process and result in higher concentrations of the hydrocarbon in the fermentation off-gas which aids in hydrocarbon recovery. Moreover, by operating at hyperbaric pressure, while the oxygen concentration will be less than 10 vol-% in the fermentation off-gas, the partial pressure of the oxygen will likely exceed the partial pressure of oxygen in air at atmospheric conditions (210 mbar).

The use of an inlet gas having an increased pressure before introduction into the fermenter of about 1.5 bar to about 15 bar (about 150 kPa to about 1500 kPa) in the process of the invention is furthermore advantageous because it facilitates a subsequent step of recovering or isolating the hydrocarbon obtained in the fermentation off-gas, which step is typically conducted under elevated (above atmospheric) pressure. The hydrocarbon can be recovered or isolated from the fermentation off-gas using techniques known in the art, such as, e.g., physical absorption with or without distillation, reactive absorption with or without distillation, adsorption, condensation, cryogenic technologies, and/or membrane-based separation. Preferably, the hydrocarbon is recovered or isolated from the fermentation off-gas using physical absorption with distillation. If, for example, the hydrocarbon to be produced in the process of the invention is isobutene, the subsequent recovery/isolation of isobutene from the fermentation off-gas is preferably carried out under a pressure of about 4 bar or higher (about 400 kPa or higher). Thus, if the total pressure of the inlet gas before introduction into the fermenter is about 4 bar (about 400 kPa) in the process of the invention and the hydrocarbon to be produced is isobutene, the resulting fermentation off-gas can be further processed in order to isolate the desired isobutene without having to compress the fermentation off-gas. This is particularly advantageous since the fermentation off-gas containing the desired hydrocarbon in combination with oxygen is potentially inflammable and should therefore ideally not need to be further compressed for subsequent isolation. Accordingly, the process of the invention using an inlet gas at a pressure before introduction into the fermenter of about 1.5 bar to about 15 bar (about 150 kPa to about 1500 kPa) is advantageous in that a further, potentially hazardous compression of the fermentation off-gas is not necessary or is required only to a lesser extent in order to isolate or purify the desired hydrocarbon from the fermentation off-gas. Moreover, compressing the inlet gas to a total pressure of about 1.5 bar to about 15 bar (about 150 kPa to about 1500 kPa) is also advantageous in terms of cost effectiveness as it avoids the need for compressing a potentially larger volume of fermentation off-gas.

An additional advantage of the elevated pressure of the inlet gas before introduction into the fermenter of about 1.5 bar to about 15 bar (about 150 kPa to about 1500 kPa) consists in that the vaporization of gaseous intermediates which may be formed in the metabolic pathway leading to the desired hydrocarbon is minimized. Accordingly, the loss of such gaseous intermediates by evaporation into the fermentation off-gas can be prevented or reduced, and a subsequent recovery or isolation of the desired hydrocarbon from the fermentation off-gas can be facilitated as there will be no or less gaseous intermediate in the fermentation off-gas. A corresponding exemplary gaseous intermediate is acetone which is formed in a metabolic pathway leading to isobutene, as also described, e.g., in van Leeuwen B N et al., Appl Microbiol Biotechnol, 2012, 93(4):1377-1387 and in WO 2011/032934. The latter reference, WO 2011/032934, describes an enzymatic method for the production of 3-hydroxy-3-methylbutyric acid (also referred to as beta-hydroxyisovalerate or HIV) from acetone and a compound which provides an activated acetyl group; the 3-hydroxy-3-methylbutyric acid thus produced can be further converted into isobutene via an enzymatically catalyzed decarboxylation reaction, as described, e.g., in WO 2010/001078 or in WO 2012/052427.

The inlet gas comprises oxygen and, while its composition is not particularly limited, it should preferably not contain any components which would compromise the capability of the cultured microorganism to produce the desired hydrocarbon. The concentration of oxygen in the inlet gas is preferably in the range of about 10 vol-% to about 40 vol-%, more preferably in the range of about 15 vol-% to about 40 vol-%, even more preferably in the range of about 17 vol-% to about 40 vol-%, even more preferably in the range of about 19 vol-% to about 35 vol-%, even more preferably in the range of about 21 vol-% to about 35 vol-%, and yet even more preferably in the range of about 21% vol-% to about 30 vol-%. The concentration of carbon dioxide in the inlet gas is preferably less than or equal to about 10 vol-%, more preferably less than or equal to about 5 vol-%, even more preferably less than or equal to about 600 vol-ppm, even more preferably less than or equal to about 400 vol-ppm, and yet even more preferably less than or equal to about 350 vol-ppm, with the minimum concentration of carbon dioxide in the inlet gas being, e.g., about 35 vol-ppm or more.

The inlet gas may, for example, be selected from (i) air, (ii) a gas mixture comprising about 78 vol-% nitrogen and about 21 vol-% oxygen, (iii) a gas mixture comprising nitrogen and about 15 vol-% to about 40 vol-% oxygen, wherein the concentration of nitrogen and oxygen in the gas mixture together makes up about 95 vol-% or more, (iv) a gas mixture comprising nitrogen and about 17 vol-% to about 40 vol-% oxygen, wherein the concentration of nitrogen and oxygen in the gas mixture together makes up about 95 vol-% or more, (v) a gas mixture comprising nitrogen and about 19 vol-% to about 35 vol-% oxygen, wherein the concentration of nitrogen and oxygen in the gas mixture together makes up about 95 vol-% or more, (vi) a gas mixture comprising nitrogen and about 21 vol-% to about 35 vol-% oxygen, wherein the concentration of nitrogen and oxygen in the gas mixture together makes up about 95 vol-% or more, (vii) a gas mixture comprising nitrogen and about 21 vol-% to about 30 vol-% oxygen, wherein the concentration of nitrogen and oxygen in the gas mixture together makes up about 95 vol-% or more, (viii) a gas mixture comprising nitrogen, about 15 vol-% to about 40 vol-% oxygen, and less than or equal to about 10 vol-% carbon dioxide, wherein the concentration of nitrogen, oxygen and carbon dioxide in the gas mixture together makes up about 95 vol-% or more, (ix) a gas mixture comprising nitrogen, about 17 vol-% to about 40 vol-% oxygen, and less than or equal to about 10 vol-% carbon dioxide, wherein the concentration of nitrogen, oxygen and carbon dioxide in the gas mixture together makes up about 95 vol-% or more, (x) a gas mixture comprising nitrogen, about 19 vol-% to about 35 vol-% oxygen, and less than or equal to about 5 vol-% carbon dioxide, wherein the concentration of nitrogen, oxygen and carbon dioxide in the gas mixture together makes up about 95 vol-% or more, (xi) a gas mixture comprising nitrogen, about 21 vol-% to about 35 vol-% oxygen, and less than or equal to about 5 vol-% carbon dioxide, wherein the concentration of nitrogen, oxygen and carbon dioxide in the gas mixture together makes up about 95 vol-% or more, or (xii) a gas mixture comprising nitrogen, about 21 vol-% to about 30 vol-% oxygen, and less than or equal to about 5 vol-% carbon dioxide, wherein the concentration of nitrogen, oxygen and carbon dioxide in the gas mixture together makes up about 95 vol-% or more. The carbon dioxide concentrations in the above-mentioned gas mixtures (viii) to (xii) can be obtained, e.g., by mixing air or oxygen-enriched air with fermentation off-gas from which the hydrocarbon to be produced has been completely or partly removed, as further described in the following.

Fermentation off-gas from which the desired hydrocarbon has been completely or partly isolated/removed can also be recycled back into the inlet gas to be introduced into the fermenter. This can be advantageous since the oxygen concentration and, accordingly, the partial pressure of oxygen in the inlet gas can thereby be decreased in a particularly cost-effective manner. In the process of the present invention, the inlet gas may thus comprise recycled fermentation off-gas from which the desired hydrocarbon has been isolated/removed. The recycled fermentation off-gas is preferably fermentation off-gas from which at least 70 vol-%, more preferably at least 80 vol-%, even more preferably at least 90 vol-%, and yet even more preferably at least 95 vol-% of the hydrocarbon originally obtained in the fermentation off-gas has been isolated/removed. For instance, any of the above-mentioned gas mixtures (i) to (vii) can be admixed—e.g., in a proportion of 20:80 (volume/volume), 30:70 (volume/volume), 40:60 (volume/volume), 50:50 (volume/volume), 60:40 (volume/volume), 70:30 (volume/volume), or 80:20 (volume/volume)—with residual fermentation off-gas from which the hydrocarbon to be produced has been completely or partly isolated/removed (e.g., fermentation off-gas from which at least 70 vol-%, preferably at least 80 vol-%, more preferably at least 90 vol-%, and even more preferably at least 95 vol-% of the hydrocarbon originally obtained in the fermentation off-gas has been isolated/removed) in order to obtain the inlet gas to be used in the process of the invention.

In particular, fermentation off-gas from which the hydrocarbon to be produced has been completely or partly isolated/removed (as described above) can be admixed to air in such an amount that the resulting gas mixture, which is used as the inlet gas in the process of the invention, contains about 15 vol-% to about 20 vol-% oxygen, preferably about 17 vol-% to about 20 vol-% oxygen. Accordingly, in the process of the present invention, the inlet gas may be a mixture of air and recycled fermentation off-gas from which the hydrocarbon has been isolated/removed, the mixture comprising nitrogen, about 15 vol-% to about 20 vol-% oxygen, and less than or equal to about 10 vol-% carbon dioxide, wherein the concentration of nitrogen, oxygen and carbon dioxide in the gas mixture together makes up about 95 vol-% or more. Preferably, the inlet gas is a mixture of air and recycled fermentation off-gas from which the hydrocarbon has been isolated/removed, the mixture comprising nitrogen, about 17 vol-% to about 20 vol-% oxygen, and less than or equal to about 10 vol-% carbon dioxide, wherein the concentration of nitrogen, oxygen and carbon dioxide in the gas mixture together makes up about 95 vol-% or more. More preferably, the inlet gas is a mixture of air and recycled fermentation off-gas from which the hydrocarbon has been isolated/removed, the mixture comprising nitrogen, about 17 vol-% to about 20 vol-% oxygen, and less than or equal to about 5 vol-% carbon dioxide, wherein the concentration of nitrogen, oxygen and carbon dioxide in the gas mixture together makes up about 95 vol-% or more. The “recycled fermentation off-gas from which the hydrocarbon has been isolated/removed”, as referred to in this paragraph, is preferably fermentation off-gas from which at least 70 vol-%, more preferably at least 80 vol-%, even more preferably at least 90 vol-%, and yet even more preferably at least 95 vol-% of the hydrocarbon originally obtained in the fermentation off-gas has been isolated/removed.

The use of air as the inlet gas is advantageous in terms of cost effectiveness. Accordingly, the inlet gas preferably comprises air, particularly at a concentration of equal to or greater than about 70 vol-%, more preferably at a concentration of equal to or greater than about 90 vol-%, and even more preferably at a concentration of equal to or greater than about 95 vol-%. Yet even more preferably, the inlet gas is air (i.e., consists of air). Depending on the metabolic pathway by which the microorganism produces the desired hydrocarbon, it may also be advantageous to use air having an increased concentration of oxygen (e.g., about 21 vol-% to about 30 vol-% oxygen) as the inlet gas.

The inlet gas described herein is preferably the only gas that is fed into the fermenter (i.e., into the liquid fermentation medium of the fermenter) throughout the process of the invention. Accordingly, it is preferred that no other gases besides the inlet gas is introduced into the fermenter's liquid fermentation medium in the process of the invention. While it is possible to add an inert gas (such as, e.g., nitrogen) to the fermentation off-gas, e.g., by feeding the inert gas directly into the headspace of the fermenter, it is preferred that no other gases are added to the fermentation off-gas obtained in the headspace of the fermenter, particularly not before the desired hydrocarbon is isolated from the fermentation off-gas.

The partial pressure of oxygen comprised in the inlet gas before introduction into the fermenter is preferably about 315 mbar to about 5.25 bar (about 31.5 kPa to about 525 kPa) and is more preferably about 315 mbar to about 4.5 bar (about 31.5 kPa to about 450 kPa). If the total pressure of the inlet gas before introduction into the fermenter is about 1.5 bar to about 8 bar (about 150 kPa to about 800 kPa), then the partial pressure of oxygen comprised in the inlet gas before introduction into the fermenter is preferably about 315 mbar to about 2.8 bar (about 31.5 kPa to about 280 kPa) and is more preferably about 315 mbar to about 2.4 bar (about 31.5 kPa to about 240 kPa). If the total pressure of the inlet gas before introduction into the fermenter is about 3.5 bar to about 6 bar (about 350 kPa to about 600 kPa), the partial pressure of oxygen comprised in the inlet gas before introduction into the fermenter is preferably about 735 mbar to about 2.1 bar (about 73.5 kPa to about 210 kPa) and is more preferably about 735 mbar to about 1.8 bar (about 73.5 kPa to about 180 kPa).

The partial pressure of carbon dioxide comprised in the inlet gas before introduction into the fermenter is preferably about 52.5 μbar to about 1.5 bar (about 5.25 Pa to about 150 kPa), more preferably about 52.5 μbar to about 9 mbar (about 5.25 Pa to about 900 Pa), and even more preferably about 52.5 μbar to about 6 mbar (about 5.25 Pa to about 600 Pa). If the total pressure of the inlet gas before introduction into the fermenter is about 1.5 bar to about 8 bar (about 150 kPa to about 800 kPa), then the partial pressure of carbon dioxide comprised in the inlet gas before introduction into the fermenter is preferably about 0.0525 mbar to about 0.8 bar (about 5.25 Pa to about 80 kPa), more preferably about 0.0525 mbar to about 4.8 mbar (about 5.25 Pa to about 480 Pa), even more preferably about 0.0525 mbar to about 3.2 mbar (about 5.25 Pa to about 320 Pa), and yet even more preferably about 0.0525 mbar to about 2.8 mbar (about 5.25 Pa to about 280 Pa). If the total pressure of the inlet gas before introduction into the fermenter is about 3.5 bar to about 6 bar (about 350 kPa to about 600 kPa), the partial pressure of carbon dioxide comprised in the inlet gas before introduction into the fermenter is preferably about 0.1225 mbar to about 0.6 bar (about 12.25 Pa to about 60 kPa), more preferably about 0.1225 mbar to about 3.6 mbar (about 12.25 Pa to about 360 Pa), even more preferably about 0.1225 mbar to about 2.4 mbar (about 12.25 Pa to about 240 Pa), and yet even more preferably about 0.1225 mbar to about 2.1 mbar (about 12.25 Pa to about 210 Pa).

As explained above, the pressure of the fermentation off-gas will be essentially the same as the pressure of the inlet gas before introduction into the fermenter. In the process of the invention, it is thus preferred to control the pressure of the fermentation off-gas to be about 1.5 bar to about 10 bar (about 150 kPa to about 1000 kPa). It may also be controlled to be, e.g., about 2 bar to about 10 bar (about 200 kPa to about 1000 kPa). More preferably, the pressure of the fermentation off-gas is controlled to be about 1.5 bar to about 8 bar (about 150 kPa to about 800 kPa), even more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). This can be achieved by adjusting the pressure of the inlet gas before introduction into the fermenter accordingly.

The fermentation off-gas containing the desired gaseous hydrocarbon in combination with oxygen is potentially combustible and therefore hazardous. In particular, as the inlet gas comprising oxygen is fed into the fermenter and travels up from the bottom sparger system to the top of the fermenter, it picks up the desired hydrogen (e.g., isobutene) produced by the microorganism in the liquid fermentation medium and, at a certain point, it will enter the flammability envelope and will become a flammable mixture (due to the high contents of oxygen and of the hydrocarbon). This gas mixture exists as very fine bubbles surrounded by the liquid fermentation medium which forms an extremely large heat sink that prevents any combustion of the gas mixture while it is dispersed in the liquid medium. Once this gas mixture exits the liquid fermentation medium (as the fermentation off-gas), however, it no longer sees the heat sink of the liquid fermentation medium and will be potentially inflammable.

The process of the invention is advantageous in that it eliminates or greatly reduces the risk of combustion of the fermentation off-gas by controlling the concentration of oxygen in the fermentation off-gas to be below about 10 vol-% and, thus, below the minimum oxygen concentration (MOC) of the hydrocarbon required for combustion. MOC values of hydrocarbons are described in the literature (e.g., in Zabetakis M G, Flammability Characteristics of Combustible Gases and Vapors, U.S. Department of the Interior, Bureau of Mines, Bulletin 627, 1965) and are mostly in the range of 10 vol-% to 11.5 vol-% oxygen. Accordingly, the control of the oxygen concentration in the fermentation off-gas in the process according to the invention advantageously improves the safety of the process and facilitates subsequent processing of the fermentation off-gas.

A lower maximum concentration of oxygen in the fermentation off-gas is preferable as it further reduces the risk of combustion and thereby increases safety. Thus, in the process according to the invention the concentration of oxygen in the fermentation off-gas is preferably controlled to be below about 8 vol-%, more preferably below about 7 vol-%, and even more preferably to be equal to or below about 6 vol-%. These maximum concentrations of oxygen in the fermentation off-gas are considerably lower than the corresponding concentrations obtained in conventional aerobic fermentation processes which typically amount to about 18 vol-% oxygen in the fermentation off-gas.

The minimum concentration of oxygen in the fermentation off-gas is not particularly limited. However, it is economically advantageous to control the oxygen concentration in the fermentation off-gas to be greater than about 1 vol-% or, even more advantageously, to be greater than about 3 vol-%. Thus, it is preferred that the concentration of oxygen in the fermentation off-gas is controlled to be equal to or greater than about 1 vol-%, more preferably equal to or greater than about 2 vol-%, and even more preferably equal to or greater than about 3 vol-%. It is particularly preferred that the concentration of oxygen in the fermentation off-gas is controlled to be about 2 vol-% to about 8 vol-%, more preferably to be about 3 vol-% to about 7 vol-%, and most preferably to be about 4 vol-% to about 6 vol-%.

If the inlet gas which is fed into the fermenter is a gas mixture comprising about 15 vol-% to about 40 vol-% oxygen (e.g., air, diluted air, or oxygen-enriched air), it will pick up the hydrocarbon produced by the microorganism as it moves through the liquid fermentation medium and will eventually become a flammable mixture (which, as explained above, is contained by the liquid fermentation medium forming an extremely large heat sink). At the same time, as this gas mixture moves through the liquid fermentation medium, its oxygen concentration will decrease due to the oxygen consumption of the microorganism. The gas mixture moving through the liquid fermentation medium will thus leave the flammability envelope at some point and will exit the liquid fermentation medium as a fermentation off-gas having an oxygen concentration below about 10 vol-%, or even lower (as described above). The inlet gas which is fed into the fermenter and takes up the desired hydrocarbon thus passes through the flammability envelope as the concentration of the hydrocarbon in the gas mixture increases and the concentration of oxygen decreases. The process according to the present invention is therefore different from known approaches which, in order to eliminate the risk of flammability of the fermentation off-gas, focus on staying outside of the flammability envelope throughout the course of the fermentation (e.g., US 2013/0164809). Such approaches, however, entail considerably higher operating costs than the process of the present invention.

The above-described control of the oxygen concentration in the fermentation off-gas can be realized, for example, by adjusting the flow rate of the inlet gas and/or by adjusting the agitation rate of the liquid fermentation medium in the fermenter and/or by adjusting the partial pressure of oxygen in the inlet gas. In accordance with the present invention, the concentration of oxygen in the fermentation off-gas can be controlled by any one or more of these measures. It is particularly preferred that the concentration of oxygen in the fermentation off-gas is controlled by adjusting the flow rate of the inlet gas and/or by adjusting the agitation rate of the liquid fermentation medium in the fermenter.

The flow rate of the inlet gas corresponds to the volume of inlet gas which is fed into the fermenter per unit time. If the flow rate of the inlet gas is increased, the oxygen concentration in the fermentation off-gas will also increase because more oxygen comprised in the inlet gas will pass through the fermenter without being consumed by the microorganism, and vice versa. Typical inlet gas flow rates may, for example, lie in the range of about 0.05 vvm to about 0.5 vvm (vessel volumes per minute, i.e. volume of inlet gas flow under standard conditions (101.325 kPa, 20° C.) per volume of liquid fermentation medium per minute) and will depend, inter alia, on the sugar concentration in the fermentation medium, the concentration of the microorganism in the medium, the reaction pathway requirements, and the specific rate of sugar consumption per unit concentration of microorganism per unit time.

The agitation rate of the liquid fermentation medium can be increased by raising the speed of an agitator stirring the medium. A higher agitation rate will increase the transfer of oxygen from the inlet gas into the liquid fermentation medium and will thus decrease the oxygen concentration in the fermentation off-gas. Conversely, reducing the agitation rate of the liquid fermentation medium will increase the oxygen concentration in the fermentation off-gas. An agitation rate of at least about 0.15 kWh per 1000 L liquid fermentation medium is generally preferred in order to allow for a favorable oxygen transfer into the liquid medium. Accordingly, the agitation rate may be, for example, about 0.15 to about 4 kWh per 1000 L liquid fermentation medium.

The partial pressure of oxygen in the inlet gas can be adjusted in different ways, e.g., by adjusting the oxygen concentration (vol-% O₂) in the inlet gas and/or by adjusting the total pressure of the inlet gas before introduction into the fermenter. In the latter case, the adjustment of the partial pressure of oxygen in the inlet gas is effected by increasing or decreasing the total pressure of the inlet gas before introduction into the fermenter. A higher total pressure of the inlet gas before introduction into the fermenter will lead to a correspondingly increased system pressure in the fermenter and will thereby increase the transfer of oxygen from the inlet gas into the liquid fermentation medium, resulting in a decreased oxygen concentration in the fermentation off-gas. Also, increasing the concentration of oxygen dissolved in the liquid fermentation medium may stimulate the growth of the microorganism and the fermentative production of the hydrocarbon by the microorganism, and may thereby effect an additional decrease in the oxygen concentration in the fermentation off-gas. Conversely, lowering the total pressure of the inlet gas before introduction into the fermenter will result in a less pronounced transfer of oxygen from the inlet gas into the liquid fermentation medium and, thus, in an increase in the oxygen concentration in the fermentation off-gas.

As described above, the partial pressure of oxygen in the inlet gas can also be adjusted by increasing or decreasing the oxygen concentration in the inlet gas. This can be achieved, e.g., by mixing or co-feeding the inlet gas with another gas having either a lower or a higher oxygen concentration than the inlet gas (for example, by admixing an inert gas, such as nitrogen or argon, or fermentation off-gas from which the desired hydrocarbon product has been partly or completely removed, to decrease the oxygen concentration in the inlet gas, or by admixing oxygen-enriched air or pure oxygen to increase the oxygen concentration in the inlet gas). The adjustment of the oxygen concentration in the inlet gas by admixing an inert gas is less advantageous in terms of cost effectiveness. The oxygen concentration in the inlet gas can also be adjusted by either removing a fraction of an inert gas (e.g., nitrogen) from the inlet gas using a corresponding membrane (e.g., a nitrogen removal membrane) or removing a fraction of oxygen from the inlet gas using an oxygen removal membrane. Suitable gas separation membranes are described, e.g., in Koros W J et al., Journal of Membrane Science, 1993, 83(1):1-80; Koros W J et al., Journal of Membrane Science, 2000, 175(2):181-196; Robeson L M, Current Opinion in Solid State and Materials Science, 1999, 4(6):549-552; Yampolskii Y, Macromolecules, 2012, 45(8):3298-3311; Baker R W, Ind Eng Chem Res, 2002, 41(6):1393-1411; Bernardo P et al., Ind Eng Chem Res, 2009, 48(10):4638-4663; and Ismail A F et al., Journal of Membrane Science, 2001, 193(1):1-18.

It will be appreciated that the concentration of oxygen in the fermentation off-gas will also depend on further factors, including the chemistry/stoichiometry of the production of the hydrocarbon by the microorganism, particularly the amount of oxygen required per molecule of the hydrocarbon, as well as the yield of the hydrocarbon per unit of sugar (e.g., glucose) consumed, and the rate at which the sugar is consumed by the microorganism per unit time (as also reflected by the oxygen uptake rate, OUR). Taking into consideration these further factors can facilitate the implementation of the process according to the invention. For example, determining the global rate of hydrocarbon (product) formation per unit time and the amount of oxygen needed per unit of hydrocarbon formed provides the rate of oxygen consumption per unit time, which will facilitate the adjustment of the inlet gas flow rate required at a given oxygen concentration in the inlet gas to attain the desired oxygen concentration in the fermentation off-gas.

In the process according to the present invention, the oxygen consumption by the microorganism (i.e., the ratio of the oxygen content in the inlet gas minus the oxygen content in the fermentation off-gas to the oxygen content in the inlet gas) is preferably equal to or greater than about 20%, more preferably it is equal to or greater than about 30%, and even more preferably it is equal to or greater than about 40%. Once the global oxygen requirement by the microorganism per unit time is determined, a person skilled in the art can make use of standard mass transfer correlations (such as the Van't Riet equation, i.e. equation 8 given in Van't Riet K, Ind Eng Chem Process Des Dev, 1979, 18(3):357-364) to define mixing intensities that, in combination with specific oxygen-carrying inlet gas parameters of flow rate, oxygen concentrations and pressure, will result in the desired minimum oxygen consumption and maximum oxygen outlet concentration in the fermentation off-gas.

In particular, based on the global rate of hydrocarbon (product) formation per unit time, the amount of oxygen needed per unit of hydrocarbon formed, and the oxygen partial pressure and flow rate of the inlet gas, the skilled person can compute the oxygen partial pressure of the off-gas via a material balance. Thus, one adjusts the inlet gas flow rate to arrive at the desired percentage oxygen consumption and oxygen partial pressure in the fermentation off-gas. To ensure that this oxygen consumption will actually occur in the fermenter, one adjusts the pressure of the inlet gas before introduction into the fermenter and the agitation rate of the liquid fermentation medium to ensure an adequate mass transfer coefficient and concentration driving force to meet the global oxygen mass transport requirement.

A high oxygen consumption is advantageous because it facilitates the adjustment of the oxygen concentration in the fermentation off-gas to desirably low values, such as below about 10 vol-%. Moreover, the more oxygen is consumed per unit volume of inlet gas, the smaller the required amount of inlet gas will be. Thus, a greater oxygen consumption allows to obtain the desired hydrocarbon in the fermentation off-gas at higher concentrations, which will make a subsequent step of recovering/separating the hydrocarbon from the fermentation off-gas more cost-effective and easier to accomplish.

In practice, a skilled person can determine the global oxygen requirement based on metabolic pathway chemistry, cell density and specific rate information, specify the concentration of oxygen in the inlet gas, decide on the concentration of oxygen to be allowed in the fermentation off-gas, and then derive the corresponding inlet gas flow rate from a material balance based on the aforementioned information. The total amount of oxygen fed into the fermenter equals the amount of oxygen consumed by the microorganism in the fermenter plus the total amount of oxygen that exits the fermenter in the fermentation off-gas, which is a theoretical material balance, i.e. the microorganism will consume that much oxygen only if it is provided with that much oxygen. In order to put this into practice, sufficient mass transfer of oxygen from the gas phase to the liquid phase is required. Therefore, once a specific pressure of the inlet gas before introduction into the fermenter and a specific dissolved oxygen concentration in the liquid fermentation medium are defined, the agitation rate required to ensure that enough oxygen is transferred from the gas phase to the liquid phase can be derived, e.g., using the Van't Riet equation.

The fermenter to be used in the process of the invention is preferably a plug flow fermenter (also referred to as a plug flow designed fermenter). Accordingly, it is preferred that at least 70%, more preferably at least 90%, and most preferably 100% of the total number of agitators that are present in the fermenter are agitators which induce radial mixing (perpendicular to the gas flow direction) and significantly reduced vertical/axial mixing (in the gas flow direction). Such agitators to be used in a plug flow fermenter can, for example, be Rushton turbine type agitators. The fermenter may thus comprise at least two (preferably at least three, more preferably at least four, even more preferably at least five) Rushton turbine type agitators which can be arranged vertically on an agitator shaft, and it is preferred that at least 70%, more preferably at least 90%, and most preferably 100% of the total number of agitators that are present in the fermenter are Rushton turbine type agitators. Rushton turbine type agitators are typically in the form of a flat disk with blades, particularly half pipe blades, that are vertically mounted and provide excellent radial mixing (perpendicular to the agitator shaft and to the gas flow direction) and significantly reduced vertical/axial mixing (in the gas flow direction), thereby creating two mixing zones per agitator. Thus, when the inlet gas comprising oxygen is fed into a plug flow fermenter (preferably at the bottom of the fermenter), it will form a gas phase dispersed in the liquid fermentation medium which will move up towards the headspace of the fermenter like a series of thin “plugs”, i.e. essentially without vertical mixing.

As a result of using a plug flow fermenter (i.e., a plug flow designed fermenter) in the process of the invention, there will be essentially no back-mixing of the gas phase moving through the liquid fermentation medium (which is formed from the inlet gas fed into the fermenter) and, consequently, the gas phase dispersed in the liquid fermentation medium will be highly staged. Towards the top of the fermenter, the oxygen concentration of the gas phase dispersed in the liquid fermentation medium will gradually decrease because of the consumption of oxygen by the cultured microorganism and because of the prevention of back-mixing of the gas phase. The use of a plug flow fermenter will thus enhance the reduction of the oxygen concentration in the fermentation off-gas (as compared to the oxygen concentration in the inlet gas) which results from the oxygen consumption by the cultured microorganism. This is highly advantageous in the process of the present invention as it greatly facilitates controlling the oxygen concentration in the fermentation off-gas to be below about 10 vol-% (or, e.g., below about 8, 7 or 6 vol-%, as described above).

The mixing concept of a plug flow fermenter is fundamentally different from the agitator design commonly used in fermentative production processes in which, e.g., one Rushton turbine type agitator may be used on the bottom of the fermenter and 3 to 5 pitched blade turbines may be placed along a vertical axis above the Rushton turbine type agitator. Such a conventional agitator design aims at maximizing radial as well as vertical/axial mixing in order to maximize the transport of oxygen from the inlet gas into the liquid fermentation medium and maintain a high oxygen content in the liquid fermentation medium. In such fermenters, the gas phase moving through the liquid fermentation medium will have an essentially uniform composition along the gas flow direction in the fermenter. In contrast thereto, the use of a plug flow fermenter (i.e., a plug flow designed fermenter) in the process of the present invention does not maximize the oxygen transport into the liquid fermentation medium by maximizing the oxygen concentration in the gas phase throughout the fermenter but, rather, it allows to maximize the microorganism-induced reduction of the oxygen concentration in the fermentation off-gas.

It is thus preferred that the fermenter to be used in the process of the invention is a plug flow fermenter (i.e., a plug flow designed fermenter) or, in other words, it is preferred that the gas flow in the fermenter is staged in such a way that it closely approximates plug flow. An example of such staging is depicted in FIG. 6-24 shown in Paul E L, Atiemo-Obeng V, and Kresta S M (eds.), Handbook of Industrial Mixing: Science and Practice, John Wiley & Sons, Inc., 2004. In contrast thereto, conventional fermentation processes typically use agitation devices that lead to significant back-mixing of the gas phase dispersed in the liquid fermentation medium. Examples of such agitation devices can be seen in FIGS. 5-25, 6-20 and 11-2 of the above-mentioned reference “Handbook of Industrial Mixing”.

An additional advantage of the plug flow fermenter is that the CO₂ partial pressure is also staged which leads to a lower bicarbonate level in the fermenter than in the case of using a backmixed fermenter. In the case of a backmixed fermenter, the concentration of CO₂ (in vol-%) is largely constant and the CO₂ partial pressure actually rises the further one goes down in the liquid fermentation medium, i.e. toward the sparger. The opposite is true of a plug flow fermenter. The concentration of CO₂ (vol-%) and the partial pressure of CO₂ are the lowest at the sparger region and rise as the bubble rises. The average partial pressure of CO₂ when using air as the inlet gas in a plug flow fermenter is about half that in a backmixed fermenter. This results in lower bicarbonate levels in the liquid fermentation medium since bicarbonate chemistry is slow relative to liquid blend times and is in equilibrium with the average and not the exit CO₂ partial pressure.

The hydrocarbon to be produced in accordance with the present invention is obtained in gaseous form in the fermentation off-gas. Accordingly, the hydrocarbon to be produced is preferably a hydrocarbon which, when present as a pure compound, has a boiling point of less than 120° C. at 1 bar (100 kPa).

Preferably, the hydrocarbon to be produced is a compound selected from class IA flammable liquids (i.e., a compound having a flash point below 73° F. (22.8° C.) and a boiling point below 100° F. (37.8° C.)), a compound selected from class IB flammable liquids (i.e., a compound having a flash point below 73° F. (22.8° C.) and a boiling point greater than or equal to 100° F. (37.8° C.)), a compound selected from class IC flammable liquids (i.e., a compound having a flash point greater than or equal to 73° C. (22.8° C.) and below 100° F. (37.8° C.)), or a compound selected from class II combustible liquids (i.e., a compound having a flash point greater than or equal to 100° F. (37.8° C.) and below 140° F. (60° C.)), as defined in the U.S. National Fire Protection Association's publication “NFPA 30, Flammable and Combustible Liquids Code, 2012 edition” (including the definitions of flash point and boiling point provided therein). More preferably, the hydrocarbon is a compound selected from class IA flammable liquids, class IB flammable liquids, or class IC flammable liquids.

It is particularly preferred that the hydrocarbon is an alkene, and more preferably a C₂₋₈ alkene, such as, e.g., a C₂₋₈ alkene of the following formula (I):

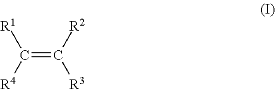

wherein R¹, R², R³ and R⁴ are each independently selected from hydrogen, C₁₋₆ alkyl or C₂₋₆ alkenyl, provided that the total number of carbon atoms in the compound of formula (I) is an integer of 2 to 8, preferably an integer of 2 to 6 (i.e., 2, 3, 4, 5 or 6), and more preferably an integer of 3 to 5 (i.e., 3, 4 or 5).

Thus, the hydrocarbon to be produced by the microorganism is preferably an alkene selected from ethene, propene, butene (e.g., isobutene, 1-butene or 2-butene), pentene, hexene, heptene, octene, butadiene (e.g., 1,3-butadiene), pentadiene (e.g., isoprene or 2-methyl-1,3-butadiene), hexadiene, heptadiene, or octadiene. More preferably, the hydrocarbon is an alkene selected from propene, butene (e.g., isobutene, 1-butene or 2-butene), pentene, butadiene (e.g., 1,3-butadiene), or pentadiene (e.g., isoprene or 2-methyl-1,3-butadiene). Even more preferably, the hydrocarbon is an alkene selected from propene, isobutene, 1-butene, 2-butene (e.g., cis-2-butene, trans-2-butene, or a mixture thereof), 1,3-butadiene, or isoprene. Even more preferably, the hydrocarbon is an alkene selected from propene, isobutene, 1,3-butadiene, or isoprene. Yet even more preferably, the hydrocarbon is isobutene or 1,3-butadiene, and most preferably it is isobutene.

In a particularly preferred embodiment, the hydrocarbon to be produced is isobutene and the total pressure of the inlet gas before introduction into the fermenter is about 3.5 bar to about 6 bar (about 350 kPa to about 600 kPa), which facilitates subsequent isolation of the isobutene from the fermentation off-gas, e.g., by physical absorption. In another specific embodiment, the hydrocarbon to be produced is propene and the total pressure of the inlet gas before introduction into the fermenter is about 14 bar to about 15 bar (about 1400 kPa to about 1500 kPa), which facilitates subsequent isolation of the propene from the fermentation off-gas, e.g., by physical absorption.

In the process of the present invention, the hydrocarbon to be produced is obtained in a gaseous state in the fermentation off-gas. Accordingly, the total pressure of the inlet gas before introduction into the fermenter and the temperature of the liquid fermentation medium will be selected depending, inter alia, on the physicochemical properties of the hydrocarbon to be produced. For example, as a general rule, the greater the number of carbon atoms comprised in the hydrocarbon, the lower the total pressure of the inlet gas before introduction into the fermenter will be. Thus, if the hydrocarbon to be produced is propene, a particularly preferred total pressure of the inlet gas before introduction into the fermenter will be about 5 bar to about 15 bar. If the hydrocarbon to be produced is isobutene, a particularly preferred total pressure of the inlet gas before introduction into the fermenter will be about 3 bar to about 5 bar. If the hydrocarbon to be produced is isoprene, a particularly preferred total pressure of the inlet gas before introduction into the fermenter will be about 2 bar to about 4 bar. If the hydrocarbon to be produced is hexene, a particularly preferred total pressure of the inlet gas before introduction into the fermenter will be about 1.5 bar to about 3 bar.

The microorganism to be used in the process of the invention may be any microorganism capable of being cultured in a fermentation medium, such as, e.g., bacterial cells, fungal cells, animal cells, or plant cells. Preferably, the microorganism to be used in accordance with the invention is a bacterium or a fungus. The bacterium is preferably selected from the genus Escherichia, Zymomonas, Rhodococcus, Pseudomonas, Bacillus, Lactobacillus, Enterococcus, Pediococcus, Alcaligenes, Klebsiella, Paenibacillus, Arthrobacter, Corynebacterium, or Brevibacterium. More preferably, the bacterium is selected from Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Pediococcus pentosaceus, Pediococcus acidilactici, Zymomonas mobilis, Corynebacterium glutamicum, or Bacillus subtilis, and even more preferably the bacterium is E. coli. The fungus is preferably a yeast, and is more preferably selected from the genus Saccharomyces, Pichia, Candida, Hansenula, Schizosaccharomyces, or Kluyveromyces. Even more preferably, the fungus or yeast is selected from Saccharomyces cerevisiae, Pichia pastoris, Candida albicans, Hansenula polymorpha, Schizosaccharomyces pombe, Kluyveromyces marxianus, or Kluyveromyces lactis. Yet even more preferably, the fungus/yeast is S. cerevisiae. Accordingly, it is particularly preferred that the microorganism is E. coli or S. cerevisiae, and most preferably the microorganism is E. coli.

The microorganism preferably produces the desired hydrocarbon using oxygen in the metabolic pathway leading to the hydrocarbon and/or the microorganism preferably uses oxygen for its growth. Accordingly, the microorganism is preferably an aerobic microorganism, such as, e.g., an obligate aerobic microorganism, a facultative anaerobic microorganism, or a microaerophilic microorganism.

In a particularly preferred embodiment, the desired hydrocarbon to be produced is isobutene and the enzymatic pathway for the production of isobutene by the microorganism involves the enzymatic conversion of acetone and acetyl-CoA into 3-hydroxy-3-methylbutyric acid. This conversion can be achieved as described in WO 2011/032934. For example, the enzymatic conversion of acetone and acetyl-CoA into 3-hydroxy-3-methylbutyric acid can be achieved by employing an enzyme having the activity of a HMG CoA synthase (EC 2.3.3.10) or an enzyme having the activity of a C—C bond cleavage/condensation lyase, such as a HMG CoA lyase (EC 4.1.3.4), or a PksG protein. In a preferred embodiment, a HMG CoA synthase (EC 2.3.3.10) is employed for this conversion. The 3-hydroxy-3-methylbutyric acid can then be further converted into isobutene in two steps. The first step, i.e. the reaction of 3-hydroxy-3-methylbutyric acid with ATP to form 3-methyl-3-phosphonoxy-butyric acid (3-phosphonoxy-isovaleric acid), is preferably achieved by an enzymatically catalyzed phosphorylation reaction as described, e.g., in WO 2012/052427. The second step is preferably achieved by an enzymatically catalyzed decarboxylation reaction as described, e.g., in WO 2010/001078 or in WO 2012/052427. It is particularly preferred that the decarboxylation to provide isobutene is achieved by using a mevalonate diphosphate decarboxylase. The microorganism is thus preferably E. coli which is able to synthesize acetone, to convert it into 3-hydroxy-3-methylbutyric acid, and to convert the 3-hydroxy-3-methylbutyric acid into isobutene (preferably by a phosphorylation and a decarboxylation reaction).

In a further preferred embodiment, the desired hydrocarbon to be produced is propene and the enzymatic pathway for the production of propene by the microorganism involves as a first step the enzymatic conversion of acetone into propan-2-ol by a secondary alcohol dehydrogenase (Hanai et al., Appl Environ Microbiol, 2007, 73:7814-7818) and as a subsequent step the dehydration of propan-2-ol to propene by a hydratase enzyme as described, e.g., in WO 2011/076691 and WO 2011/076689. It is preferred that the dehydration of propan-2-ol into propene is achieved by an oleate hydratase (EC 4.2.1.53), kievitone hydratase (EC 4.2.1.95), or phaseollidin hydratase (EC 4.2.1.97).

In a further preferred embodiment, the desired hydrocarbon to be produced is 1,3-butadiene and the enzymatic pathway for the production of 1,3-butadiene by the microorganism involves the enzymatic conversion of 2-buten-1-ol (crotyl alcohol) or 3-buten-2-ol into 1,3-butadiene. The conversion of 3-buten-2-ol into 1,3-butadiene is preferably achieved by an enzymatically catalyzed dehydration reaction. It is preferred that the conversion is achieved by using an oleate hydratase (EC 4.2.1.53), kievitone hydratase (EC 4.2.1.95), or phaseollidin hydratase (EC 4.2.1.97). The conversion of 2-buten-1-ol (crotyl alcohol) into 1,3-butadiene is preferably achieved by using a crotyl alcohol kinase, a 2-butenyl-4-phosphate kinase, and a butadiene synthase, as described, e.g., in WO 2012/106516.

In a further preferred embodiment, the desired hydrocarbon to be produced is isoprene and the enzymatic pathway for the production of isoprene by the microorganism involves the enzymatic conversion of 3-methyl-2-buten-1-ol (prenol), 2-methyl-3-buten-2-ol, 3-methyl-3-buten-1-ol (isoprenol) or 3-methyl-3-buten-2-ol into isoprene. These conversions are preferably achieved by an enzymatically catalyzed dehydration reaction. It is preferred that the conversion is achieved by using an oleate hydratase (EC 4.2.1.53), kievitone hydratase (EC 4.2.1.95), or phaseollidin hydratase (EC 4.2.1.97).

The process of the present invention is carried out by culturing the microorganism in a liquid fermentation medium. A fermentation medium is used which allows the growth of the microorganism to be cultured and the production of the hydrocarbon to be produced. The choice of an appropriate culture medium belongs to the common general knowledge of the person skilled in the art.

Generally, a fermentation medium contains a suitable carbon source. Examples of suitable carbon sources are, but are not limited to, monosaccharides such as glucose or fructose, oligosaccharides such as lactose or sucrose, polysaccharides such as starch or cellulose or mixtures thereof, and unpurified mixtures from renewable feedstocks such as cheese whey permeate, cornsteep liquor, sugar beet molasses, and barley malt.

The carbon source may also be a one-carbon substrate, such as carbon dioxide or methanol, for which metabolic conversion into key biochemical intermediates has been demonstrated. For example, glycerol production from single carbon sources (e.g., methanol, formaldehyde or formate) has been reported in methylotrophic yeasts (Yamada K et al., Agric Biol Chem, 1989, 53:541-543) and in bacteria (Hunter et al., Biochemistry, 1985, 24:4148-4155). These organisms can assimilate single carbon compounds, ranging in oxidation state from methane to formate, and produce glycerol. The pathway of carbon assimilation can be through ribulose monophosphate, through serine, or through xylulose-monophosphate (Gottschalk, Bacterial Metabolism, Second Edition, Springer-Verlag, New York, 1986). The ribulose monophosphate pathway involves the condensation of formate with ribulose-5-phosphate to form a six-carbon sugar that becomes fructose and eventually the three-carbon product glyceraldehyde-3-phosphate. Likewise, the serine pathway assimilates the one-carbon compound into the glycolytic pathway via methylenetetrahydrofolate.

Some organisms, such as methylotrophic organisms, are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb Growth C1 Compd, [Int. Symp.], 7th (1993), 415-432. Editor(s): Murrell, J. Collin; Kelly, Don P. Publisher: Intercept, Andover, UK). Similarly, various species of Candida metabolize alanine or oleic acid (Sulter et al., Arch Microbiol, 1990, 153:485-489). Therefore, the carbon source contained in the liquid fermentation medium in the process according to the present invention may encompass a wide variety of carbon containing substrates and will only be limited by the choice of microorganism and possibly by the choice of the desired hydrocarbon to be produced.

Preferred carbon sources comprised in the liquid fermentation medium are glucose, fructose, sucrose, sugar mixtures derived from cellulosic or lignocellulosic feedstocks, or methanol. More preferably, the liquid fermentation medium comprises glucose as a carbon source.

The carbon source (e.g., glucose) may be provided in the liquid fermentation medium at a concentration of, e.g., about 1 g/l to about 20 g/l, particularly about 5 g/l to about 15 g/l, or alternatively about 10 g/l.

In addition to an appropriate carbon source, the fermentation medium generally also contains suitable minerals, salts, cofactors, buffers and other components, known to those skilled in the art, which are suitable for the growth of the microorganism and the promotion of the enzymatic pathway necessary for the production of the desired hydrocarbon.

The fermentation medium to be used is not critical but it must support the growth of the microorganism used and promote the biosynthetic pathway necessary to produce the desired hydrocarbon. A conventional fermentation medium may be used, including, but not limited to: complex media containing organic nitrogen sources such as yeast extract or peptone and at least one fermentable carbon source; minimal media; and defined media. Preferred growth media in the present invention are common commercially prepared media such as Luria Bertani (LB) broth, Sabouraud Dextrose (SD) broth or Yeast Medium (YM) broth. Other defined or synthetic growth media may also be used and the appropriate medium for growth of the particular microorganism will be known by a person skilled in the art of microbiology or fermentation science. The pH value of the fermentation medium is adjusted to meet the growth requirements of the microorganism employed in the process. Suitable pH ranges for the fermentation are generally between pH 5.0 to pH 9.0, where pH 6.0 to pH 8.0 is preferred as the initial condition.

The microorganism is cultured in the liquid fermentation medium at a suitable temperature depending on the requirements of the particular microorganism. Typical fermentation conditions employ temperatures of about 25° C. to about 45° C., preferably about 30° C. to about 37° C. However, if thermophilic microorganisms are employed in the process of the invention, higher temperatures may be used.

The process according to the present invention may be carried out in any suitable fermenter. Such fermenters include, e.g., a stirred tank fermenter, an airlift fermenter, a bubble column fermenter, or any combination thereof. As described above, the fermenter is preferably a plug flow fermenter (i.e., a plug flow designed fermenter). The fermenter to be used in the process of the invention (e.g., a stirred tank fermenter or, in particular, a plug flow fermenter) may optionally be equipped with a pH electrode, an oxygen sensor electrode, a carbon dioxide sensor electrode, a glucose sensor electrode (e.g., an enzymatic glucose sensor electrode or a non-enzymatic glucose sensor electrode), and/or a temperature control unit.

Typically and preferably, the process of the invention is carried out as a batch process. A classical batch fermentation is a closed system where the composition of the media is set at the beginning of the fermentation and is not subject to artificial alterations during the fermentation. Thus, at the beginning of the fermentation the medium is inoculated with the desired microorganism or microorganisms and fermentation is permitted to occur without adding anything to the system. Typically, however, a “batch” fermentation is batch process with respect to the addition of the carbon source and attempts are often made at controlling factors such as pH and oxygen concentration. In batch systems the metabolite and biomass compositions of the system change constantly up to the time the fermentation is stopped. Within batch cultures microorganism cells moderate through a static lag phase to a high growth log phase and finally to a stationary phase where growth rate is diminished or halted. If untreated, cells in the stationary phase will eventually die. Cells in log phase generally are responsible for the bulk of the hydrocarbon production.

Most preferably, the process according to the present invention is carried out as a fed-batch process, i.e. the microorganism is cultured in a liquid fermentation medium in a fermenter under fed-batch conditions. A fed-batch process comprises a typical batch system with the exception that the substrate is added in increments as the fermentation progresses. Fed-batch systems are particularly useful when catabolite repression is apt to inhibit the metabolism of the microorganism cells and where it is desirable to have limited amounts of substrate in the media. Batch and fed-batch fermentations are common and well known in the art and examples may be found in Thomas D. Brock in Biotechnology: A Textbook of Industrial Microbiology, Second Edition (1989), Sinauer Associates, Inc., Sunderland, Mass., or in Deshpande, Mukund V., Appl Biochem Biotechnol, 1992, 36, 227.

Although the present invention is preferably carried out in batch mode (and most preferably in fed-batch mode), it is in principle also possible that the process is carried out using continuous fermentation. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth. One advantage of continuous fermentation is that it allows for the modulation of one factor or any number of factors that affect cell growth or end product concentration. By continuous fermentation it is attempted to maintain steady state growth conditions and thus the cell loss due to media being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock (loc. cit.).

In a particularly preferred embodiment, the hydrocarbon to be produced in the process according to the invention is a C₂₋₈ alkene and the concentration of oxygen in the inlet gas is in the range of about 15 vol-% to about 40 vol-%. In this embodiment, the C₂₋₈ alkene is preferably selected from ethene, propene, isobutene, 1-butene, 2-butene, 1,3-butadiene, or isoprene, and is more preferably isobutene. The concentration of oxygen in the inlet gas is preferably in the range of about 17 vol-% to about 40 vol-%, more preferably in the range of about 19 vol-% to about 35 vol-%, and even more preferably in the range of about 21 vol-% to about 35 vol-%. Yet even more preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 10 bar (about 150 kPa to about 1000 kPa), more preferably about 1.5 bar to about 8 bar (about 150 kPa to about 800 kPa), even more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be below about 8 vol-%, and is more preferably controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is a C₂₋₈ alkene and the concentration of oxygen in the inlet gas is in the range of about 17 vol-% to about 40 vol-%. In this embodiment, the C₂₋₈ alkene is preferably selected from ethene, propene, isobutene, 1-butene, 2-butene, 1,3-butadiene, or isoprene, and is more preferably isobutene. The concentration of oxygen in the inlet gas is preferably in the range of about 19 vol-% to about 35 vol-%, and more preferably in the range of about 21 vol-% to about 35 vol-%. Even more preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 10 bar (about 150 kPa to about 1000 kPa), more preferably about 1.5 bar to about 8 bar (about 150 kPa to about 800 kPa), even more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be below about 8 vol-%, and is more preferably controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is a C₂₋₈ alkene, the concentration of oxygen in the inlet gas is in the range of about 15 vol-% to about 40 vol-%, and the total pressure of the inlet gas before introduction into the fermenter is about 1.5 bar to about 10 bar (about 150 kPa to about 1000 kPa). In this embodiment, the C₂₋₈ alkene is preferably selected from ethene, propene, isobutene, 1-butene, 2-butene, 1,3-butadiene, or isoprene, and is more preferably isobutene. The concentration of oxygen in the inlet gas is preferably in the range of about 17 vol-% to about 40 vol-%, more preferably in the range of about 19 vol-% to about 35 vol-%, and even more preferably in the range of about 21 vol-% to about 35 vol-%. Yet even more preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 8 bar (about 150 kPa to about 800 kPa), more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be below about 8 vol-%, and is more preferably controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is a C₂₋₈ alkene, the concentration of oxygen in the inlet gas is in the range of about 17 vol-% to about 40 vol-%, and the total pressure of the inlet gas before introduction into the fermenter is about 1.5 bar to about 10 bar (about 150 kPa to about 1000 kPa). In this embodiment, the C₂₋₈ alkene is preferably selected from ethene, propene, isobutene, 1-butene, 2-butene, 1,3-butadiene, or isoprene, and is more preferably isobutene. The concentration of oxygen in the inlet gas is preferably in the range of about 19 vol-% to about 35 vol-%, and more preferably in the range of about 21 vol-% to about 35 vol-%. Even more preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 8 bar (about 150 kPa to about 800 kPa), more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be below about 8 vol-%, and is more preferably controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is selected from ethene, propene, isobutene, 1-butene, 2-butene, 1,3-butadiene, or isoprene, the concentration of oxygen in the inlet gas is in the range of about 17 vol-% to about 40 vol-%, the total pressure of the inlet gas before introduction into the fermenter is about 1.5 bar to about 10 bar (about 150 kPa to about 1000 kPa), and the concentration of oxygen in the fermentation off-gas is controlled to be below about 8 vol-%. In this embodiment, the hydrocarbon to be produced is preferably isobutene. The concentration of oxygen in the inlet gas is preferably in the range of about 19 vol-% to about 35 vol-%, and more preferably in the range of about 21 vol-% to about 35 vol-%. Even more preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 8 bar (about 150 kPa to about 800 kPa), more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is selected from ethene, propene, isobutene, 1-butene, 2-butene, 1,3-butadiene, or isoprene, the concentration of oxygen in the inlet gas is in the range of about 17 vol-% to about 40 vol-%, the total pressure of the inlet gas before introduction into the fermenter is about 1.5 bar to about 8 bar (about 150 kPa to about 800 kPa), and the concentration of oxygen in the fermentation off-gas is controlled to be below about 8 vol-%. In this embodiment, the hydrocarbon to be produced is preferably isobutene. The concentration of oxygen in the inlet gas is preferably in the range of about 19 vol-% to about 35 vol-%, and more preferably in the range of about 21 vol-% to about 35 vol-%. Even more preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 6 bar (about 150 kPa to about 600 kPa), more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is selected from ethene, propene, isobutene, 1-butene, 2-butene, 1,3-butadiene, or isoprene, the concentration of oxygen in the inlet gas is in the range of about 19 vol-% to about 35 vol-%, the total pressure of the inlet gas before introduction into the fermenter is about 1.5 bar to about 10 bar (about 150 kPa to about 1000 kPa), and the concentration of oxygen in the fermentation off-gas is controlled to be below about 8 vol-%. In this embodiment, the hydrocarbon to be produced is preferably isobutene. The concentration of oxygen in the inlet gas is preferably in the range of about 21 vol-% to about 35 vol-%. More preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 8 bar (about 150 kPa to about 800 kPa), more preferably about 1.5 bar to about 6 bar (about 150 kPa to about 600 kPa), even more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is selected from ethene, propene, isobutene, 1-butene, 2-butene, 1,3-butadiene, or isoprene, the concentration of oxygen in the inlet gas is in the range of about 19 vol-% to about 35 vol-%, the total pressure of the inlet gas before introduction into the fermenter is about 1.5 bar to about 8 bar (about 150 kPa to about 800 kPa), and the concentration of oxygen in the fermentation off-gas is controlled to be below about 8 vol-%. In this embodiment, the hydrocarbon to be produced is preferably isobutene. The concentration of oxygen in the inlet gas is preferably in the range of about 21 vol-% to about 35 vol-%. More preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 1.5 bar to about 6 bar (about 150 kPa to about 600 kPa), more preferably about 2 bar to about 6 bar (about 200 kPa to about 600 kPa), even more preferably about 3 bar to about 6 bar (about 300 kPa to about 600 kPa), and yet even more preferably about 3.5 bar to about 6 bar (about 350 kPa to about 600 kPa). In this embodiment, it is furthermore preferred that the concentration of oxygen in the fermentation off-gas is controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

In a further particularly preferred embodiment, the hydrocarbon to be produced in the process according to the invention is isobutene, the concentration of oxygen in the inlet gas is in the range of about 19 vol-% to about 35 vol-%, the total pressure of the inlet gas before introduction into the fermenter is about 1.5 bar to about 6 bar (about 150 kPa to about 600 kPa), and the concentration of oxygen in the fermentation off-gas is controlled to be below about 8 vol-%. In this embodiment, the concentration of oxygen in the inlet gas is preferably in the range of about 21 vol-% to about 35 vol-%. More preferably, the inlet gas is a gas mixture comprising nitrogen and about 21 vol-% to about 35 vol-% oxygen, wherein the concentration of nitrogen and oxygen in the gas mixture together makes up about 95 vol-% or more. The total pressure of the inlet gas before introduction into the fermenter is preferably about 2 bar to about 6 bar (about 200 kPa to about 600 kPa), more preferably about 3 bar to about 6 bar (about 300 kPa to about 600 kPa), and even more preferably about 3.5 bar to about 6 bar (about 350 kPa to about 600 kPa). The concentration of oxygen in the fermentation off-gas is preferably controlled to be equal to or below about 6 vol-%. The fermenter is preferably a plug flow fermenter. The microorganism is preferably E. coli. The present invention specifically relates to each and every combination of the aforementioned general and/or preferred features of this embodiment.

As used herein, the term “hydrocarbon” refers to a compound which consists of carbon atoms and hydrogen atoms and may be saturated or unsaturated, linear, branched or cyclic, aliphatic or aromatic.

The term “alkene” refers to an unsaturated aliphatic (i.e., non-aromatic) acyclic hydrocarbon which may be linear or branched and comprises at least one (e.g., one or two) carbon-to-carbon double bond while it does not comprise any carbon-to-carbon triple bond. A “C₂₋₈ alkene” refers an alkene having 2 to 8 carbon atoms. Specifically included in this definition are also alkenes having two carbon-to-carbon double bonds (i.e., alkadienes), such as, e.g., 1,3-butadiene or isoprene.

The term “alkyl” refers to a monovalent saturated aliphatic acyclic hydrocarbon group (i.e., a group consisting of carbon atoms and hydrogen atoms) which may be linear or branched and does not comprise any carbon-to-carbon double bond or any carbon-to-carbon triple bond.

The term “alkenyl” refers to a monovalent unsaturated aliphatic acyclic hydrocarbon group which may be linear or branched and comprises at least one carbon-to-carbon double bond while it does not comprise any carbon-to-carbon triple bond.

The term “fermentative production” refers to the production of a compound by a micoorganism in culture.

The term “about” refers to ±10% of the indicated numerical value, and in particular to ±5% of the indicated numerical value. Whenever the term “about” is used, a specific reference to the exact numerical value indicated is also included. For example, the expression “about 100” refers to the range of 90 to 110, in particular the range of 95 to 105, and preferably refers to the specific value of 100.

It is to be understood that the present invention specifically relates to each and every combination of features and process parameters described herein, including any combination of general and/or preferred features/parameters. In particular, the invention specifically relates to all combinations of preferred features (including all degrees of preference) of the process provided herein.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following examples which are merely illustrative and are not to be construed as a limitation of the scope of the present invention.

### EXAMPLES

### Example 1

### Fermentative Production of Isobutene from Glucose (at 4 Bar)

This example is meant to be illustrative but not inclusive of the various means to convert glucose to isobutene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 absorbance units (AU) is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 4 bar absolute throughout the course of the fermentation.

The air flow rate is set at such a rate that the exit oxygen concentration is always less than the about 13% level that is the flammable minimum oxygen concentration (MOC) value for isobutene in the presence of CO₂ and N₂ mixtures (Zabetakis M G, Flammability Characteristics of Combustible Gases and Vapors, U.S. Department of the Interior, Bureau of Mines, Bulletin 627, 1965, p. 52). Typically the exit concentration is maintained below 8%. Based on the chemistry of the reaction this corresponds to an air flow rate of less than 1,700 standard liters/minute of air at the end of the fermentation cycle. Agitation intensity is maintained such as to ensure that the utilization of the oxygen fed to the fermenter is greater than 45%. Typically this means that the agitation intensity will be about 0.7 KW during the early stage of the fermentation cycle (i.e., microbe growth phase) and will rise to 7 KW by the end of the fermentation cycle.

Due to isobutene's low solubility in water (about 260 ppm at 1 bar and 20° C.), isobutene is continuously vaporized and continuously exits with the fermentation off-gases throughout the course of the fermentation. Isobutene does not accumulate to any appreciable extent in the fermentation liquid.

Due to acetone's higher boiling point, while it is also continuously venting into the fermentation off-gases, it also accumulates in the fermentation broth. Since the acetone conversion step is slow and a high rate depends on a high acetone concentration, this is advantageous to overall fermenter productivity. During the course of the initial microbe growth phase, acetone will accumulate to a 20 to 50 grams/liter concentration. An equilibrium between accumulation and consumption/venting will be reached and the acetone level will be maintained in this range during the rest of the fermentation cycle. The fermentation will continue until either contamination or the build-up of inhibitory compounds causes a notable decrease in overall isobutene production rate. This typically occurs in the 60th to 80th hour of the cycle.

### Example 2

### Fermentative Production of 1,3-Butadiene from Glucose (at 3 Bar)

This example is meant to be illustrative but not inclusive of the various means to convert glucose to 1,3-butadiene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 AU is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 3 bar absolute throughout the course of the fermentation.

The air flow rate is set at such a rate that the exit oxygen concentration is always less than the about 10.5% level that is the flammable minimum oxygen concentration (MOC) value for 1,3-butadiene in the presence of CO₂ and N₂ mixtures (Zabetakis M G, Flammability Characteristics of Combustible Gases and Vapors, U.S. Department of the Interior, Bureau of Mines, Bulletin 627, 1965, p. 55). Typically the exit concentration is maintained below 8%. Based on the chemistry of the reaction this corresponds to an air flow rate of less than 1,100 standard liters/minute of air at the end of the fermentation cycle. Agitation intensity is maintained such as to ensure that the utilization of the oxygen fed to the fermenter is greater than 45%. Typically this means that the agitation intensity will be about 0.4 KW during the early stage of the fermentation cycle (i.e., microbe growth phase) and will rise to 6 KW by the end of the fermentation cycle.

Due to 1,3-butadiene's low solubility in water (about 735 ppm at 1 bar and 20° C.), 1,3-butadiene is continuously vaporized and continuously exits with the fermentation off-gases throughout the course of the fermentation. 1,3-Butadiene does not accumulate to any appreciable extent in the fermentation liquid.

Due to crotyl alcohol's higher boiling point, while it is also continuously venting into the fermentation off-gases, it also accumulates in the fermentation broth. Since the crotyl alcohol conversion step is slow and a high rate depends on a high crotyl alcohol concentration, this is advantageous to overall fermenter productivity. During the course of the initial microbe growth phase, crotyl alcohol will accumulate to about 5 to 10 grams/liter concentration. An equilibrium between accumulation and consumption/venting will be reached and the crotyl alcohol level will be maintained in this range during the rest of the fermentation cycle. The fermentation will continue until either contamination or the build-up of inhibitory compounds causes a notable decrease in overall 1,3-butadiene production rate. This typically occurs in the 60th to 80th hour of the cycle.

### Example 3

### Fermentative Production of 1,3-Butadiene from Glucose (at 2 Bar)

This example is meant to be illustrative but not inclusive of the various means to convert glucose to 1,3-butadiene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 absorbance units (AU) is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide and/or caustic addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 2 bar absolute throughout the course of the fermentation.

The air flow rate is set at such a rate that the exit oxygen concentration is always less than the about 10.5% level that is the flammable minimum oxygen concentration (MOC) value for 1,3-butadiene in the presence of CO₂ and N₂ mixtures (Zabetakis M G, Flammability Characteristics of Combustible Gases and Vapors, U.S. Department of the Interior, Bureau of Mines, Bulletin 627, 1965, p. 55). Typically the exit concentration is maintained below 8%. Based on the chemistry of the reaction this corresponds to an air flow rate of less than 1,100 standard liters/minute of air at the end of the fermentation cycle. Agitation intensity is maintained such as to ensure that the utilization of the oxygen fed to the fermenter is greater than 45%. Typically this means that the agitation intensity will be about 0.4 KW during the early stage of the fermentation cycle (i.e., microbe growth phase) and will rise to 14 KW by the end of the fermentation cycle.

Due to 1,3-butadiene's low solubility in water (about 735 ppm at 1 bar and 20° C.), 1,3-butadiene is continuously vaporized and continuously exits with the fermentation off-gases throughout the course of the fermentation. 1,3-Butadiene does not accumulate to any appreciable extent in the fermentation liquid.

Due to crotyl alcohol's higher boiling point, while it is also continuously venting into the fermentation off-gases, it also accumulates in the fermentation broth. Since the crotyl alcohol conversion step is slow and a high rate depends on a high crotyl alcohol concentration, this is advantageous to overall fermenter productivity. During the course of the initial microbe growth phase, crotyl alcohol will accumulate to about 5 to 10 grams/liter concentration. An equilibrium between accumulation and consumption/venting will be reached and the crotyl alcohol level will be maintained in this range during the rest of the fermentation cycle. The fermentation will continue until either contamination or the build-up of inhibitory compounds causes a notable decrease in overall 1,3-butadiene production rate. This typically occurs in the 60th to 80th hour of the cycle.

### Example 4

### Fermentative Production of Propene from Glucose (at 3 Bar)

This example is meant to be illustrative but not inclusive of the various means to convert glucose to propene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 absorbance units (AU) is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide and/or caustic addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 3 bar absolute throughout the course of the fermentation.

The air flow rate is set at such a rate that the exit oxygen concentration is always less than the about 13% level that is the flammable minimum oxygen concentration (MOC) value for propylene in the presence of CO₂ and N₂ mixtures (Zabetakis M G, Flammability Characteristics of Combustible Gases and Vapors, U.S. Department of the Interior, Bureau of Mines, Bulletin 627, 1965, p. 51). Typically the exit concentration is maintained below 4%. Based on the chemistry of the reaction this corresponds to an air flow rate of less than 560 standard liters/minute of air at the end of the fermentation cycle. Agitation intensity is maintained such as to ensure that the utilization of the oxygen fed to the fermenter is greater than 55%. Typically this means that the agitation intensity will be about 0.4 KW during the early stage of the fermentation cycle (i.e., microbe growth phase) and will rise to 7.5 KW by the end of the fermentation cycle.

Due to propylene's low solubility in water (about 400 ppm at 1 bar and 20° C.), propylene is continuously vaporized and continuously exits with the fermentation off-gases throughout the course of the fermentation. Propylene does not accumulate to any appreciable extent in the fermentation liquid.

Due to acetone's and isopropanol's higher boiling points, while they are also continuously venting into the fermentation off-gases, they also accumulate in the fermentation broth. Since the acetone and isopropanol conversion steps are not instantaneous, a high rate depends on high acetone and isopropanol concentrations; this is advantageous to overall fermenter productivity. During the course of the initial microbe growth and early production phases, acetone will accumulate to about 10-15 grams/liter concentration and isopropanol will accumulate to about 20-30 grams/liter concentration. An equilibrium between accumulation and consumption/venting will be reached and the acetone and isopropanol levels will be maintained in this range during the rest of the fermentation cycle.

### Example 5

### Fermentative Production of Propene from Glucose (at 10 Bar)

This example is meant to be illustrative but not inclusive of the various means to convert glucose to propene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 absorbance units (AU) is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide and/or caustic addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 10 bar absolute throughout the course of the fermentation.

The air flow rate is set at such a rate that the exit oxygen concentration is always less than the about 13% level that is the flammable minimum oxygen concentration (MOC) value for propylene in the presence of CO₂ and N₂ mixtures (Zabetakis M G, Flammability Characteristics of Combustible Gases and Vapors, U.S. Department of the Interior, Bureau of Mines, Bulletin 627, 1965, p. 51). Typically the exit concentration is maintained below 1%. Based on the chemistry of the reaction this corresponds to an air flow rate of less than 380 standard liters/minute of air at the end of the fermentation cycle. Agitation intensity is maintained such as to ensure that the utilization of the oxygen fed to the fermenter is greater than 85%. Typically this means that the agitation intensity will be about 0.4 KW during the early stage of the fermentation cycle (i.e., microbe growth phase) and will rise to 6.6 KW by the end of the fermentation cycle.

Due to propylene's low solubility in water (about 400 ppm at 1 bar and 20° C.), propylene is continuously vaporized and continuously exits with the fermentation off-gases throughout the course of the fermentation. Propylene does not accumulate to any appreciable extent in the fermentation liquid.

Due to acetone's and isopropanol's higher boiling points, while they are also continuously venting into the fermentation off-gases, they also accumulate in the fermentation broth. Since the acetone and isopropanol conversion steps are not instantaneous, a high rate depends on high acetone and isopropanol concentrations; this is advantageous to overall fermenter productivity. During the course of the initial microbe growth and early production phases, acetone will accumulate to about 10-15 grams/liter concentration and isopropanol will accumulate to about 20-30 grams/liter concentration. An equilibrium between accumulation and consumption/venting will be reached and the acetone and isopropanol levels will be maintained in this range during the rest of the fermentation cycle.

### Example 6

### Fermentative Production of Propene from Glucose (at 15 Bar)

This example is meant to be illustrative but not inclusive of the various means to convert glucose to propene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 absorbance units (AU) is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide and/or caustic addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 15 bar absolute throughout the course of the fermentation.

The air flow rate is set at such a rate that the exit oxygen concentration is always less than the about 13% level that is the flammable minimum oxygen concentration (MOC) value for propylene in the presence of CO₂ and N₂ mixtures (Zabetakis M G, Flammability Characteristics of Combustible Gases and Vapors, U.S. Department of the Interior, Bureau of Mines, Bulletin 627, 1965, p. 51). Typically the exit concentration is maintained below 1%. Based on the chemistry of the reaction this corresponds to an air flow rate of less than 370 standard liters/minute of air at the end of the fermentation cycle. Agitation intensity is maintained such as to ensure that the utilization of the oxygen fed to the fermenter is greater than 85%. Typically this means that the agitation intensity will be about 0.4 KW during the early stage of the fermentation cycle (i.e., microbe growth phase) and will rise to 4.2 KW by the end of the fermentation cycle.

Due to propylene's low solubility in water (about 400 ppm at 1 bar and 20° C.), propylene is continuously vaporized and continuously exits with the fermentation off-gases throughout the course of the fermentation. Propylene does not accumulate to any appreciable extent in the fermentation liquid.

Due to acetone's and isopropanol's higher boiling points, while they are also continuously venting into the fermentation off-gases, they also accumulate in the fermentation broth. Since the acetone and isopropanol conversion steps are not instantaneous, a high rate depends on high acetone and isopropanol concentrations; this is advantageous to overall fermenter productivity. During the course of the initial microbe growth and early production phases, acetone will accumulate to about 10-15 grams/liter concentration and isopropanol will accumulate to about 20-30 grams/liter concentration. An equilibrium between accumulation and consumption/venting will be reached and the acetone and isopropanol levels will be maintained in this range during the rest of the fermentation cycle.

### Example 7

### Fermentative Production of Isoprene from Glucose (at 3 Bar)

This example is meant to be illustrative but not inclusive of the various means to convert glucose to isoprene via fermentation. Materials and methods suitable for the maintenance and growth of bacterial cultures are well known in the art and will not be described in detail in this example. Techniques suitable for use can be found in Thomas D. Brock in Biotechnology: A Textbook of Industrial Microbiology, 2nd Edition (1989), Sinnauer Associates, Inc, Sunderland, Mass. Cultures are started from frozen stock in shaker flasks and grown in medium until an OD₅₅₀ of approximately 1.0 absorbance units (AU) is reached at which time the contents are transferred to a 1,000 liter seed fermenter. The microbes are further grown in the seed fermenter until they reach an OD₅₅₀ of approximately 10 AU at which time they are transferred into the 10,000 liter production fermenter.

Anti-foam, medium salts (containing phosphates, sulfate and citrates) and 6000 liters of water are sterilized in the production fermenter prior to the addition of the contents of the seed fermenter. pH is adjusted to 6.8 via ammonium hydroxide and/or caustic addition and enough concentrated glucose solution (60-70 wt %) is added to bring the glucose concentration to 10 grams/liter. Glucose concentration is maintained at 0 to 10 grams/liter throughout the fermentation. Temperature is controlled at 32° C. The pressure in the head space of the fermenter is maintained at 3 bar absolute throughout the course of the fermentation.

The air flow rate is set at such a rate that the exit oxygen concentration is always less than the about 9.7% level that is the flammable minimum oxygen concentration (MOC) value for isoprene in the presence of CO₂ and N₂ mixtures as given in U.S. Pat. No. 8,420,360 B2. Typically the exit concentration is maintained below 5%. Based on the chemistry of the reaction this corresponds to an air flow rate of less than 750 standard liters/minute of air at the end of the fermentation cycle. Agitation intensity is maintained such as to ensure that the utilization of the oxygen fed to the fermenter is greater than 50%. Typically this means that the agitation intensity will be about 0.6 KW during the early stage of the fermentation cycle (i.e., microbe growth phase) and will rise to about 7 KW by the end of the fermentation cycle.

Due to isoprene's low solubility in water (about 642 ppm at 1 bar and 25° C.), isoprene is continuously vaporized and continuously exits with the fermentation off-gases throughout the course of the fermentation. Isoprene does not accumulate to any appreciable extent in the fermentation liquid.

### Example 8

### Effect of High Partial Pressure of Carbon Dioxide on the Growth and Metabolism of E. coli Cultured on Glucose

Escherichia coli, strain MG1655 (ATCC Number 700926; American Type Culture Collection (ATCC), Manassas, Va.), was grown in batch mode in 1.4 L Multifors fermenters (Infors A G, Basel, Switzerland) equipped with temperature, pH and dO (dissolved oxygen) control. Initially, the fermenters contained in 1 L total volume: KH₂PO₄, 7.5 g/L; MgSO₄.7H₂O, 2.0 g/L; citric acid, 1.83 g/L; ferric ammonium citrate, 0.3 g/L; CaCl₂.2H₂O, 0.2 g/L; sulfuric acid (98%), 1.2 mL/L; yeast extract, 5 g/L; and glucose (10 g/L). After sterilization, 10 mL of a trace metal mixture and 1 mL of a thiamine solution (150 mM) was added and the medium was adjusted to the desired pH (see below) with 3 M NaOH. The trace metal mixture contained: MnSO₄.H₂O, 3 g/L; NaCl, 1 g/L; FeSO₄.7H₂O, 0.1 g/L; CoCl₂.6H₂O, 0.1 g/L; ZnSO₄.7H₂O, 0.1 g/L; CuSO₄.5H₂O, 0.0064 g/L; H₃BO₃, 0.01 g/L; and Na₂MoO₄.2H₂O, 0.01 g/L. In one set of three fermenters, gas flow (air) was fixed at 0.5 VVM (volume per volume per minute) and pH was automatically maintained at pH 6.2, pH 6.5 and 6.8 with the addition of 3 M NaOH. In a second set of three fermenters, gas flow (synthetic gas, 21% O₂ and 79% CO₂) was fixed at 0.5 VVM and pH was automatically maintained at pH 6.2, pH 6.5 and 6.8 as described above. For each of the fermenters, the respective oxygen-carrying inlet gas had atmospheric pressure (about 1.013 bar) before introduction into the fermenter, temperature control was set at 37° C. and dO control was set for 20% with no back pressure. The stirrer speed was initially set at 400 rpm (1200 rpm maximum).

After equilibration with the gases, evident by the cessation of NaOH addition, the fermenters were inoculated at an initial OD₆₀₀ of approximately 0.4 absorbance units (AU). At various times after inoculation, until at least 60% of the glucose was consumed, aliquots were withdrawn for biomass, glucose, and by-product composition analyses using techniques well known to those skilled in the art.

The initial growth rate observed in the air fed fermenters was not affected by pH with growth rate (μ)=0.62±0.02 h⁻¹. The initial growth rate observed in the synthetic gas fed fermenters also was not affected by pH with growth rate (μ)=0.39±0.03 h⁻¹. The biomass yield observed in the air fed fermenters was also not affected by pH (biomass yield=0.55±0.02 gram biomass/gram glucose consumed). The biomass yield observed in the synthetic gas fed fermenters was 0.47, 0.48 and 0.35 gram biomass/gram glucose consumed for the fermenters maintained at pH 6.2, pH 6.5 and 6.8, respectively. The maximum specific glucose consumption rate for each of the fermenters was approximately 1 gram glucose consumed per gram dry cell weight per hour. The by-products produced were those typically observed with E. coli under glucose excess condition, comprising acetate, succinate, lactate, formate and ethanol.

These results demonstrate that bacteria, such as E. coli, can be cultured at a high partial pressure of carbon dioxide (pCO₂˜800 mbar) with only slight impairments of growth rate. This finding is surprising in consideration of the predominant view in the literature that partial pressures of carbon dioxide as low as pCO₂˜300 mbar significantly inhibit microbial growth (Baez A et al., Biotechnol Bioeng, 2009, 104(1):102-110), leading to the expectation of complete inhibition at pCO₂˜800 mbar. It has thus been confirmed that the process according to the invention can be realized.

1. A process for the fermentative production of a hydrocarbon, wherein said process comprises:
(a) culturing a microorganism producing the hydrocarbon in a liquid fermentation medium in a fermenter,
(b) feeding an inlet gas comprising oxygen into the fermenter wherein the total pressure of the inlet gas before introduction into the fermenter is about 1.5 bar to about 15 bar;
(c) obtaining the hydrocarbon in a gaseous state in the fermentation off-gas,
(d) controlling the concentration of oxygen in the fermentation off-gas to be below about 10 vol-%.

2. The process of claim 1, wherein the total pressure of the inlet gas before introduction into the fermenter is about 1.5 bar to about 10 bar.

3. The process of claim 1, wherein the total pressure of the inlet gas before introduction into the fermenter is about 3.5 bar to about 6 bar.

4. The process of claim 1, wherein the partial pressure of oxygen comprised in the inlet gas before introduction into the fermenter is about 315 mbar to about 5.25 bar.

5. The process of claim 1, wherein the concentration of oxygen in the inlet gas is in the range of about 15 vol-% to about 40 vol-%.

6. The process of claim 1, wherein the concentration of oxygen in the inlet gas is in the range of about 21 vol-% to about 35 vol-%.

7. The process of claim 1, wherein the partial pressure of carbon dioxide comprised in the inlet gas before introduction into the fermenter is about 52.5 μbar to about 1.5 bar.

8. The process of claim 1, wherein the inlet gas is a gas mixture comprising nitrogen and about 15 vol-% to about 40 vol-% oxygen and wherein the concentration of nitrogen and oxygen in the gas mixture together makes up about 95 vol-% or more.

9. The process of claim 1, wherein the inlet gas is a gas mixture comprising nitrogen and about 21 vol-% to about 35 vol-% oxygen and wherein the concentration of nitrogen and oxygen in the gas mixture together makes up about 95 vol-% or more.

10. The process of claim 1, wherein the inlet gas comprises air at a concentration of equal to or greater than about 90 vol-%.

11. The process of claim 1, wherein the inlet gas is air.

12. The process of claim 1, wherein the inlet gas comprises recycled fermentation off-gas from which the hydrocarbon has been isolated.

13. The process of claim 12, wherein the inlet gas comprises mixture of air and recycled fermentation off-gas from which the hydrocarbon has been isolated, the mixture comprising nitrogen, about 15 vol-% to about 20 vol-% oxygen, and less than or equal to about 10 vol-% carbon dioxide and wherein the concentration of nitrogen, oxygen and carbon dioxide in the mixture together makes up about 95 vol-% or more.

14. The process of claim 1, wherein the concentration of oxygen in the fermentation off-gas is controlled to be below about 8 vol-%.

15. The process of claim 1, wherein the concentration of oxygen in the fermentation off-gas is controlled to be about 4 vol-% to about 6 vol-%.

16. The process of claim 1, wherein the concentration of oxygen in the fermentation off-gas is controlled by adjusting the flow rate of the inlet gas and/or, by adjusting the agitation rate of the liquid fermentation medium in the fermenter and/, or by adjusting the partial pressure of oxygen in the inlet gas.

17. The process of claim 1, wherein the oxygen consumption by the microorganism is equal to or greater than about 30%.

18. The process of claim 1, wherein the fermenter is a plug flow fermenter.

19. The process of claim 18, wherein the fermenter further comprises at least two Rushton turbine type agitators.

20. The process of claim 18, wherein all agitators present in the fermenter are Rushton turbine type agitators.

21. The process of claim 1, wherein the hydrocarbon is an alkene.

22. The process of claim 1, wherein the hydrocarbon is an alkene of the following formula (I): 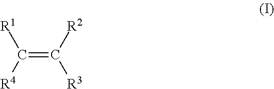
wherein R¹, R², R³ and R⁴ are each independently selected from hydrogen, C₁₋₆ alkyl or C₂₋₆ alkenyl, provided that the total number of carbon atoms in the compound of formula (I) is an integer of 2 to 8.

23. The process of claim 21, wherein the alkene is propene, isobutene, 1,3-butadiene, or isoprene.

24. The process of claim 21, wherein the alkene is isobutene.

25. The process of claim 1, wherein the microorganism is a bacterium or a fungus.

26. The process of claim 25, wherein the microorganism is Escherichia coli.

27. The process of claim 1, wherein the temperature of the liquid fermentation medium is about 30° C. to about 37° C.
